(19) United States
(12) Patent Application Publication
(10) Pub. No.: US 2006/0025788 A1
(43) Pub. Date: Feb. 2, 2006

(54) Anastomotic leg arrangement

(75) Inventors: Amir Loshakove, Moshav-Bazra (IL); Ofer Nativ, Rishon-Lezion (IL); Ido Kilemnik, Herzelia (IL); Gil Hefer, Kfar-Saba (IL)

Correspondence Address: WOLF, BLOCK, SCHORR & SOLIS-COHEN LLP, 250 PARK AVENUE, NEW YORK, NY, 10177, US

(73) Assignee: By-Pass, Inc., Orangeburg, NY (US)

(21) Appl. No.: 10/528,865

(22) PCT Filed: Sep. 25, 2003
(86) PCT No.: PCT/IL03/00769

§ 371 Date: Mar. 24, 2005

Related U.S. Application Data
(60) Provisional application No. 60/426,013, filed on Nov. 14, 2002.

(30) Foreign Application Priority Data
Sep. 25, 2002 (WO) PCT/IL02/00790

(51) Int. Cl. A61B 17/08

(52) U.S. Cl. 606/153

(57) ABSTRACT

A leg compacting system for compacting inwards a group of legs (228) of an anastomotic connector towards a central location thereof, comprising, a coupler for coupling to a delivery system on which said connector is mounted; and a plurality of leg confiners (820), said leg confiners (820) configured to selectively move in an inwards direction and said confiners configured to have a resting compacted configuration in which a space of between 7 and 1 mm in width is maintained between the innermost leg contacting sections of said confiners such that a graft vessel suitable for mounting on said connector can be passed between said confiners (820).

### RELATED APPLICATIONS

The present application claims priority from and is a continuation-in-part of PCT application PCT/IL02/00790, filed on Sep. 25, 2002, which designates the US, now published in English as WO 03/026475. It also claims priority as well as the benefit under 119 (e) of U.S. Ser. No. 60/426,013, filed on Nov. 14, 2002. The disclosure of both applications is incorporated herein by reference.

### FIELD OF THE INVENTION

The present invention relates to systems and tools used in conjunction with anastomotic connectors and/or manipulation of blood vessels.

### BACKGROUND OF THE INVENTION

Connecting together of two blood vessels may be performed using an anastomotic connector. In an end-to-side connection, such a connector typically has a final diameter similar to that of the “end” vessel. In some connectors, the end vessel is everted over the connector. An example of such a connector is shown in Kaster U.S. Pat. No. 5,234,447, the disclosure of which is incorporated herein by reference. The manipulation of the vessel to be everted may require delicate motor control by the physician and there is a risk of tearing of the vessel due to its being stretched.

Also, as shown in Kaster, the forward legs are inserted into an aperture in a blood vessel. Such exact insertion may also require delicate motor control and be time consuming and/or difficult.

### SUMMARY OF THE INVENTION

A broad aspect of the present invention relates to methods and apparatus for radially compacting groups of anastomotic legs into a radically compact configuration. In an exemplary embodiment of the invention, the groups of legs are radially compacted prior to eversion, to reduce the effective diameter of the connector at the leg group, over which a graft vessel is evereted. Optionally, a passage is maintained between the legs for the passage of a graft vessel, for example a passage of between 1 and 3 mm, or a passage of under 4 or 5 mm. Optionally, the group is radially compacted prior to insertion into an aperture in a target blood vessel.

In an exemplary embodiment of the invention, a leg compacting system is removably mounted on an anastomosis delivery system. Optionally, the compacting system has the form of a cap. Alternatively the compacting system is integrated with the delivery system, optionally having a minimal profile so as to not interfere with visibility and/or not catch on nearby tissue. Optionally, the compacting system is configured to be operated using one hand, optionally a hand already holding the delivery system.

Optionally, the leg compacting system comprises a leg receptacle for each leg. Optionally, the receptacles prevent tangling of the legs during compacting and/or releasing of the legs. Optionally, the receptacles set relative leg positions for eversion.

In an exemplary embodiment of the invention, the leg compacting system comprises a pair of opposing bars which selectively advance towards each other, trapping and compacting the legs between them. In an alternative embodiment of the invention, the leg compacting system comprises a plurality of individual leg receptacles, all of which are advanced in concert.

In an alternative exemplary embodiment of the invention, the leg compacting system comprises a splitable cap. Optionally, the cap is split using a control on its side.

In an alternative exemplary embodiment of the invention, the leg compacting system comprises a splitable tube which is torn off to release the legs.

In an alternative exemplary embodiment of the invention, the leg compacting system comprises a lasso-like element (hereafter “lasso”) formed a noose-like loop (hereafter “noose”) at its end which can be reduced in diameter. The compacting system also comprises a receptacle which contains the lasso, fits over the legs and assists in capturing the legs with the lasso. Optionally, a manual controller is provided for selectively and repeatedly releasing and compacting the legs. Optionally, the lasso comprises a single wire folded to form a loop and a tube covering at least a section of the folded wire adjacent the loop. The loop is tightened as a noose by advancing the tube relatively to the loop.

Optionally, the leg compacting system is substantially see-through, for example, being of open construction or being formed of a transparent material, so that the area of the procedure and/or the connector ends are not hidden from an operator's view.

An aspect of some embodiments of the invention relates to a leg arranging device for a noose leg compactor. In an exemplary embodiment of the invention, the leg arranging devices automatically or manually releases a pre-tensioned noose to close around legs of a connector, once the legs are properly positioned with respect to the noose.

An aspect of some embodiments of the invention relates to a noose controller. Optionally, the controller is stored in a configuration which elastically urges the noose to close. Optionally, a lock is provided to reduce stress on the noose itself. Optionally, the noose is cut at a certain position of the controller.

An aspect of some embodiments of the invention relates to the release configuration of the legs. In an exemplary embodiment of the invention, the legs are arranged in a generally ellipsoid or circular configuration. In some embodiments of the invention, releasing the legs that are near the ends of the ellipsoid first, stretches the aperture and assists in the other legs engaging blood vessel wall tissue.

An aspect of some embodiments of the invention relates to a method of mounting a graft into a capsule having an anastomosis connector loaded therein. In an exemplary embodiment of the invention, the capsule is held in a cradle, for example a leg compacting cap, and the capsule is split, so that a graft can be laid inside the capsule. The capsule is then closed and the graft can be everted on the connector legs. The capsule may then be loaded on a delivery system. In an exemplary embodiment of the invention, the connector comprises a plurality of clips, for example clips in which long legs are torn of after the long legs lock to medallions or opposing short legs. In an alternative embodiment, the connector is a ring connector in which the ring is split at a location matching the split in the capsule.

An aspect of some embodiments of the invention relates to a delivery system with a handle that can be used at various orientations to the system. In an exemplary embodiment of the invention, a handle of the delivery system is selectively rotatable and/or attachable at multiple rotational orientations. Optionally, a tip of the delivery system in which a graft is mounted, is rotatable relative to a body of the delivery system. Optionally, a trigger is used for controlling the delivery of an anastomosis connector. Optionally, the handle is devoid of controls. Optionally, a pistol grip handle is provided.

An aspect of some embodiments of the invention relates to a step-action delivery system in which the delivery system advances and retracts a connector, in addition to or instead of at least some such motions by a user. In an exemplary embodiment of the invention, the delivery system separately provides the actions of advancing the connector tip relative to the delivery system and tearing of the connector, in which no further advancing is provided. Optionally, a user can deploy an anastomosis connector without being required to move the delivery system once the legs are inserted into an aperture in the blood vessel. In an exemplary embodiment of the invention, the forces required to tear the connector are provided by releasing a spring, rather than by the user. Optionally, a single control is used for both steps.

An aspect of some embodiments of the invention relates to a anastomotic connector leg manipulation tool. In an exemplary embodiment, the transfixion device comprises a handle from which two prongs separated by a fixed distance, extend, thereby forming an aperture between the prongs adapted to at least partially encircle a connector leg. Optionally, each prong is adapted to pass between adjacent connector legs. In an exemplary embodiment of the invention, the manipulation tool is used to arrange the legs, individually, during compacting. Optionally, the tool is used to arrange the legs during eversion and/or to assist in impaling a graft vessel with a hook portion of a leg and/or to advance an everted section. Optionally, the tool is used to push legs into an aperture in a blood vessel, for example, if one or more of the legs pops out during insertion and releasing of the legs.

An aspect of some embodiments of the invention relates to using a shunt during anastomosis. In an exemplary embodiment of the invention, a shunt is inserted into a target vessel through an aperture formed therein for an anastomosis. The shunt prevent blood from flowing near the aperture and shunts it around the anastomosis location so that tissue served by the blood vessel is not starved. Optionally, the shunt serves to stretch out the blood vessel, so the aperture is not collapsed and is easily accessible. In an exemplary embodiment of the invention, the shunt is removed between legs of the connector, once the connector is properly placed.

There is thus provided in accordance with an exemplary embodiment of the invention, a leg compacting system for compacting inwards a group of legs of an anastomotic connector towards a central location thereof, comprising:
a coupler for coupling to a delivery system on which said connector is mounted; and
a plurality of leg confiners, said leg confiners configured to selectively move in an inwards direction and said confiners configured to have a resting compacted configuration in which a space of between 7 and 1 mm in width is maintained between the innermost leg contacting sections of said confiners such that a graft vessel suitable for mounting on said connector can be passed between said confiners. Optionally, said resting configuration is an innermost configuration. Optionally, said space is less than 5 mm in width.

In an exemplary embodiment of the invention, said space is oval or circular.

In an exemplary embodiment of the invention, said system is configured to be selectively dismounted from said delivery system.

In an exemplary embodiment of the invention, the system comprises a control for splitting at least a portion of said system for removal from said delivery system.

In an exemplary embodiment of the invention, said confiners move in a radial direction.

In an exemplary embodiment of the invention, the system comprises a rotatable control which selectively moves said confiners in a radial direction.

In an exemplary embodiment of the invention, said system is permanently mounted on said delivery system.

In an exemplary embodiment of the invention, each confiner is configured to receive a single leg.

In an exemplary embodiment of the invention, said plurality of leg confiners are configured to release legs when moved outwards, said release being not simultaneous for all legs.

There is also provided in accordance with an exemplary embodiment of the invention, a leg compacting system for compacting inwards a group of legs of an anastomotic connector towards a central location thereof, comprising:
a coupler for coupling to a delivery system on which said connector is mounted; and
a plurality of leg confiners, said leg confiners configured to selectively move in an inward direction and said confiners configured automatically engage said legs as they move inward inwards. Optionally, wherein each leg confiner is configured to receive a plurality of legs. Alternatively or additionally, each leg confiner is configured to receive a single leg. Alternatively or additionally, said motion is radial.

There is also provided in accordance with an exemplary embodiment of the invention, a leg compacting system for compacting inwards a group of legs of an anastomotic connector towards a central location thereof, comprising:
at least one wire arranged to selectively move inwards, from a position outwards of the legs, thereby compacting the legs; and
a controller which is operative to selectively moving said wire. Optionally, said wire is adapted to engage said legs near a hook section of the legs. Alternatively or additionally, the system comprises at least two wires configured to compact the legs simultaneously from two directions.

In an exemplary embodiment of the invention, said wire comprises a side wall adapted to prevent legs from slipping away from compacting.

There is also provided in accordance with an exemplary embodiment of the invention, a leg arranging device for use with a noose, comprising:
a body adapted to receive a delivery system on which a plurality of connector legs are mounted;
a noose receptacle arranged around an expected position of said legs, said receptacle including an inner block which selectively blocks said noose from leaving said receptacle to engage said legs; and
a control is operative removes said block. Optionally, said control is automatically activated to release said noose once a delivery system is inserted in said body and said legs are at said expected position. Alternatively, said control is manually activated.

In an exemplary embodiment of the invention, the device comprises a noose controller which pre-stresses said noose to reduce its diameter.

There is also provided in accordance with an exemplary embodiment of the invention, a leg compacting system for compacting inwards a group of legs of an anastomotic connector towards a central location thereof, comprising:
at least one element adapted to compact a plurality of connector legs to a compacted configuration; and
at least controller configured to activate said element thereby releasing said legs,
wherein, said at least one element is configured to release certain connector legs before other ones of said legs as said element is activated. Optionally, said element is configured to first release legs that are on a long axis of an incision into which said legs are inserted for connection. Alternatively or additionally, said at least one element defines a plurality of notches each configured for receiving at least one leg and wherein an end notch of said at least one element is configured to release a leg earlier than a leg held by a more central notch.

There is also provided in accordance with an exemplary embodiment of the invention, a leg compacting system for compacting inwards a group of legs of an anastomotic connector towards a central location thereof, comprising:
a cap adapted to mount on a delivery system, said cap comprising:
   at least two sections adapted to form said cap;
      each of said sections having a front plate defining a plurality of receptacles for legs; and
a control adapted to split said cap into said sections.

There is also provided in accordance with an exemplary embodiment of the invention, a method of using a noose-type compacting system, comprising:
mounting a noose on a plurality of connector legs;
compacting the legs using the noose;
inserting said compacted legs into an aperture in a blood vessel;
releasing and tightening said noose until a desired leg configuration is achieved; and
removing said noose. Optionally, removing said noose comprises cutting an extension of said noose. Alternatively or additionally, mounting comprises:
arranging said noose around said legs, such that said noose is pre-stressed; and
releasing said noose to engage said legs.

There is also provided in accordance with an exemplary embodiment of the invention, an anastomosis connector delivery system, comprising:
a body having an end adapted to mount a connector thereon;
a handle adapted to be attached to said body at a plurality of different orientation positions relative to said body;
at least one control for deploying said connector, said handle being devoid of controls for deploying the connector. Optionally, said handle is a pistol grip. Alternatively or additionally, said control is shaped as and moves as a trigger.

There is also provided in accordance with an exemplary embodiment of the invention, an anastomosis delivery system, comprising:
a body having a handle section and having a plurality of connector legs attached to one end thereof;
an activation control; and
a leg retraction and tearing mechanism,
wherein said control both retracts said legs relative to said body and tears said legs and wherein said control applies said retracting and said tearing without requiring movement of said handle by a user. Optionally, said control releases a spring which provides said tearing. Alternatively or additionally, said body comprises a moving section and a stationary section, such that said retraction of said legs moves said moving section towards said legs and does not move said legs relative to said stationary section.

In an exemplary embodiment of the invention, the system comprises a recoil absorber which absorbs at least part of a recoil of said spring prior to such recoil affecting said handle/

There is also provided in accordance with an exemplary embodiment of the invention, a sterile anastomosis connector leg manipulator, comprising:
a handle; and
a two pronged extension defining a receptacle between the prongs, said receptacle being sized to receive one leg of an anastomosis connector suitable for attaching a vessel of a diameter smaller than 4 mm, said prongs being thin enough to fit between adjacent legs of such a connector.

There is also provided in accordance with an exemplary embodiment of the invention, a leg arranging system for arranging legs of a blood vessel anastomotic connector, comprising:
a coupler for coupling to a delivery system on which said connector is mounted;
a body; and
a plurality of notches defined by said body, each of said notch configured to hold a single leg. Optionally, said notches prevent said legs from crossing. Alternatively or additionally, said notches position said legs in an inwards compacted configuration.

There is also provided in accordance with an exemplary embodiment of the invention, a method of mounting a graft, comprising:
arranging a plurality of connector legs in a leg arranger, such that their relative positions are fixed in a plane perpendicular to a general orientation of said legs;
providing a graft between said legs; and
mounting said graft on said arranged legs. Optionally, said legs are arranged to have an inward compacted configuration.

There is also provided in accordance with an exemplary embodiment of the invention, a method of inserting a graft into a blood vessel, comprising:
arranging a plurality of connector legs in a leg arranger to a have a desired mutual positional relationship and an inward compacted configuration; and
inserting said arranged legs into an aperture of a blood vessel.

There is also provided in accordance with an exemplary embodiment of the invention, an anastomotic connector for blood vessels, comprising:
a ring; and
a plurality of legs arranged around said ring, wherein at least two legs at opposing sides of said ring are configured to bend radially out more than other of said legs.

There is also provided in accordance with an exemplary embodiment of the invention, an anastomotic connector for blood vessels, comprising:
a ring; and
a plurality of legs arranged around said ring, wherein at least two legs at opposing sides of said ring are configured to be stiffer than other of said legs.

There is also provided in accordance with an exemplary embodiment of the invention, an anastomotic connector kit, comprising:
a plurality of leg segments arranged in a generally circular configuration; and
a plurality f leg locking segments, each adapted to be locked to one leg,
wherein, wherein at least two legs at opposing sides of said circle are configured to be stiffer than other of said legs.

There is also provided in accordance with an exemplary embodiment of the invention, an anastomotic connector kit, comprising:
a plurality of leg segments arranged in a generally circular configuration; and
a plurality f leg locking segments, each adapted to be locked to one leg,
wherein, wherein at least two legs at opposing sides of said circle are configured to bend radially out more than other of said legs.

There is also provided in accordance with an exemplary embodiment of the invention, a connector kit, comprising:
a sterile package
a connector having a plurality of forward legs; and
a band radially compacting said legs towards a center.

There is also provided in accordance with an exemplary embodiment of the invention, a method of mounting a graft on a connector delivery system capsule, comprising:
axially splitting said capsule;
laying said graft in said capsule;
closing said capsule; and
mounting said capsule on a connector of said capsule.

There is also provided in accordance with an exemplary embodiment of the invention, apparatus for mounting a graft on a spoilable graft capsule, comprising:
a splitable connector capsule;
a body including a receptacle large enough to hold a split capsule and including a slot in its side; and
a control which selectable opens said body so said capsule can open. Optionally, said control actively splits said capsule. Alternatively or additionally, said body is adapted to radially compact legs of a connector of said capsule.

There is also provided in accordance with an exemplary embodiment of the invention, a method of graft attachment to a target vessel, comprising:
inserting a plurality of legs into an aperture in said target vessel;
releasing at least two of said legs so that said at least two legs stretch said aperture; and
completing said anastomosis. Optionally, said releasing comprises releasing to ends of an incision.

There is also provided in accordance with an exemplary embodiment of the invention, a method of graft attachment to a target vessel, comprising:
inserting a plurality of legs into an aperture in said target vessel;
mechanically retracting said legs relative to a body of a delivery system; and
mechanically tearing said legs,
wherein said retracting and said tearing occur without motion of said legs relative to said vessel.

There is also provided in accordance with an exemplary embodiment of the invention, a method of performing an anastomosis, comprising:
forming an opening a target vessel;
inserting a shunt into said target vessel to bypass said opening;
inserting a plurality of anastomosis connector legs into said aperture;
removing said shunt between said legs, while said legs are in said aperture; and
completing said anastomosis.

### BRIEF DESCRIPTION OF THE FIGURES

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the Figures in which they appear, in which:

FIGS. 1A and 1B are schematic side views of situations where compacting of legs of an anastomotic device may be useful;

FIGS. 2 and 3 are isometric views of a bar-type leg compacting device, mounted on an anastomotic connector holder, in accordance with an exemplary embodiment of the invention;

FIGS. 4-6 are isometric and schematic views of a splitable leg compacting and arranging cap and its operation, in accordance with an exemplary embodiment of the invention;

FIGS. 7A-7F are top views of an aperture in a blood vessel demonstrating the positions of anastomotic connector legs as they are selectively released during connection to the host vessel, in accordance with an exemplary embodiment of the invention;

FIG. 8 is an isometric view of a compacting overtube, in accordance with an exemplary embodiment of the invention;

FIGS. 9-12 are schematic cross-sectional, side and isometric views of a radial leg compacting and arranging device, in accordance with an exemplary embodiments of the invention;

FIG. 13 is an isometric view of a lasso-based leg arranging device and controller, in accordance with an exemplary embodiment of the invention;

FIGS. 14A and 14B are side cross-sectional views of a lasso arranging device in two operating states, in accordance with an exemplary embodiment of the invention;

FIG. 15 is side cross-sectional views of a lasso arranging device with a connector delivery system mounted therein, in accordance with an exemplary embodiment of the invention;

FIG. 16 is a cross-sectional view of a section of a lasso controller, showing a lasso tearing mechanism, in accordance with an exemplary embodiment of the invention;

FIG. 17 is a partial cross-sectional view of an alternative embodiment of a lasso arranging system, in accordance with an exemplary embodiment of the invention;

FIG. 18 is a side cross-sectional view of a pistol-grip connector delivery system, in accordance with an exemplary embodiment of the invention;

FIG. 19 is an isometric view of the pistol-grip connector delivery system of FIG. 18, in accordance with an exemplary embodiment of the invention;

FIGS. 20A and 20B are isometric views of a leg manipulator, in accordance with an exemplary embodiment of the invention;

FIG. 21 is a partially cross-sectioned side view of the leg manipulator of FIGS. 20A and 20B, demonstrating the manipulation of a leg during eversion, in accordance with an exemplary embodiment of the invention;

FIG. 22 is a side view of the leg manipulator of FIGS. 20A and 20B, demonstrating leg manipulation during insertion of connector legs into a target vessel, in accordance with an exemplary embodiment of the invention; and

FIGS. 23A-23E illustrate the use of a shunt, in accordance with an exemplary embodiment of the invention.

### DETAILED DESCRIPTION OF EXEMPLARY EMBODIMENTS

Leg Configuration for Eversion

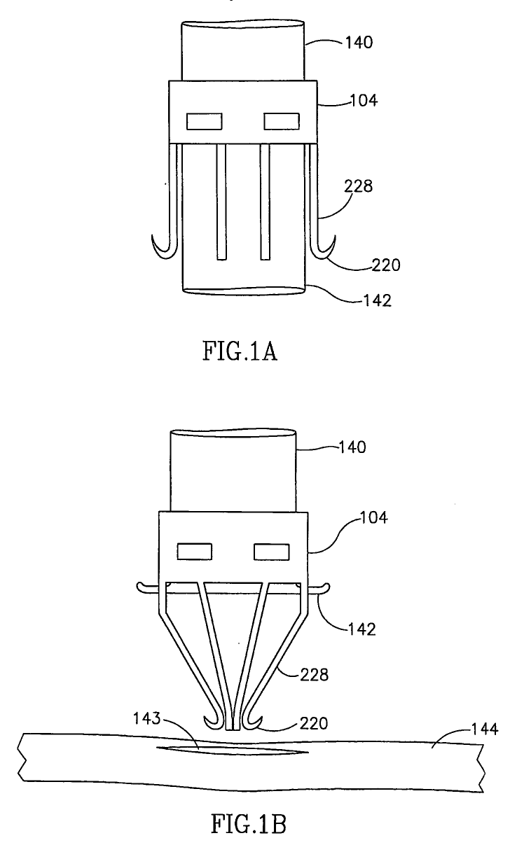

FIG. 1A shows a graft 140 being mounted on a part of an anastomotic connector 104. For clarity, the rest of the connector and/or other parts thereof and/or a delivery or holding system are not shown. To complete the mounting, a lip 142 of graft 140 needs to be everted over hooks 220 of forward legs 228. In the configuration shown, this requires stretching lip 142. In exemplary embodiments of the invention, as shown below, a leg compacting device is provided to radially compact legs 228, thereby reducing their outer radius and obviating or reducing the need to stretch lip 142.

In some embodiments of the invention, the leg compacting device also sets relative leg positions for eversion.

Leg Configuration for Insertion

FIG. 1B shows connector 104 being deployed, after a graft 140 is mounted thereon. In the type of anastomotic connection shown, hook ends 220 of legs 228 need to be inserted into an incision (or aperture) 143 in a target vessel 144. If incision 143 is made too long, it is easier to insert hooks 220 into incision 143, however, there is a danger that the anastomosis will not cover all of incision 143 and there may be a leakage of blood. If incision 143 is made smaller, it is more difficult to insert hooks 220 into it. In exemplary embodiments of the invention, as shown below, a leg compacting device brings legs 228 together, as shown in FIG. 1B, so that the outer diameter as measured at hooks 220 is smaller than the diameter of incision 143 and insertion is facilitated.

The devices used for radial leg compacting for eversion may be the same or different than those used for insertion. In addition, it should be noted that depending on the eversion method and/or delivery system one or the other of the leg compacting situations may be mooted.

Bar-Type Leg Compacting Device

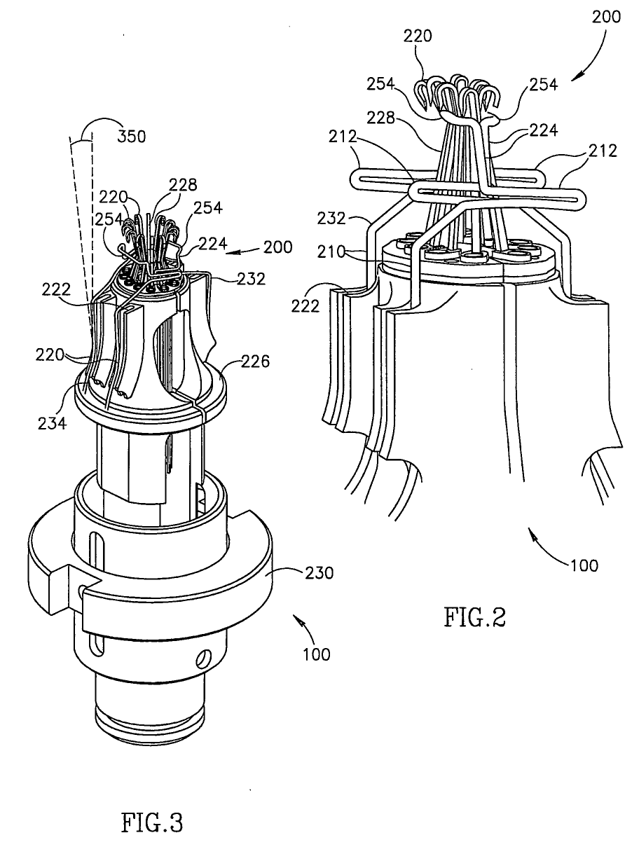

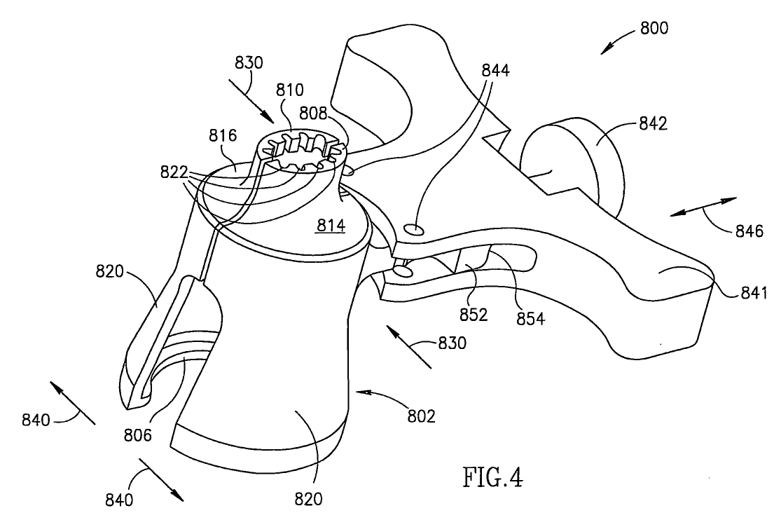

FIGS. 2 and 3 are isometric views of a bar-type leg compacting device 200, mounted on an anastomotic connector holder 100, in accordance with an exemplary embodiment of the invention. Device 200 comprises two bars 254 which oppose each other and are positioned so that legs 228 are between them. When the bars are brought together, the configuration of legs 228 is radially compacted. FIG. 2 shows legs 228 compacted by bars 254 and FIG. 3 shows legs 228 released from bars 254 and having a natural configuration. Depending on the embodiment, the released legs may be further apart and/or without a bend in them.

In the embodiment shown in FIGS. 2 and 3, a pair of opposing bars 254 radially compact legs 228, near hooks 220, to have a generally linear configuration. Compacting near hooks 220 has a potential advantage that legs 228 do not cross each other, which may cause undesirable entanglement. Alternatively or additionally, compacting is performed at a point of pre-defined bend in legs 228.

In an exemplary embodiment of the invention, compacting device 200 is used prior to insertion of hooks 220 into a blood vessel bars 254 optionally enter the blood vessel, however this is not required in some embodiments. In the configuration of FIG. 2, the hooks are generally arranged in a line which can be more easily fit into an incision, than the spread out configuration of FIG. 3. Alternatively or additionally, device 200 is used during eversion, for example before or after the graft (not shown) is everted over at least one hook 220.

In the exemplary embodiment shown, bars 254 are smooth, so that legs 228 can slip along them. In some cases, such slippage is undesirable and roughened bars 254 may be used. Alternatively or additionally, notches may be defined in the bars, to receive legs 228. The notches may be, for example, arranged to correspond to a desired leg arrangement, optionally with one notch provided per leg. Alternatively, the notches may be arranged so that they engage a leg if the leg slides past a notch.

While bars 254 are shown to be straight, in other embodiments, bars 254 may be used to define other configurations of compacted legs. For example, bars 254 may define a compressed ellipse, a bent line or be arranged so that the ends of one of bars 254 is curved away from the ends of the other bar. Each such arrangement may be suitable for particular incision shapes and/or to avoid leg entanglement problems, for example. Optionally, device 200, the head of delivery system 100, and/or the whole delivery system, are adapted for rotation to better align the legs configuration with the incision (or other aperture) in a target blood vessel. In embodiments where the connector used is not rotationally symmetric, it may be desirable to maintain a fixed relationship (or one with limited range of motion) between the orientations of bars 254 and the connector.

While two bars 254 are shown, embodiments with a higher number of bars can be used to obtain more complex configurations, for example three bars can be used to obtain a triangular configuration, with one end that may be easier to insert into a blood vessel. Also a single bar can be used, for example, if incomplete compacting is preferred, or if the bar goes past the mid-line axis of delivery system 100, so that it contacts all or most of the legs.

In the embodiments shown, a pair of sides walls 212 is provided with each bar 254 thus generally defining (from a top view) a rectangle with one wall missing. In some embodiments, as noted below, side walls 212 flare out. Optionally, side walls are used to prevent legs 228 from slipping out from between bars 254. Optionally, the side walls are used to ensure that even in the open configuration, all the legs are engagable by bars 254. It should be noted that in the embodiment shown bars 254 are slightly recessed from extenders 224 which hold them. These recess may also prevent slipping out of legs 228 during compacting. In an alternative embodiment of the invention, described below, it is desirable that some of legs 228 be released prior to other ones of the legs. In an exemplary embodiment of the invention, side walls 212 or the recession of bars 254 is reduced, to allow early release of end legs, which have a pre-defined deformation to bend out in a direction parallel to bars 254.

Optionally, extenders 224 also serve to distance side walls 212 from bars 254. Optionally, side walls 212 can thus serve to herd legs 228 to correct alignment with bars 254. Alternatively or additionally, side walls 212 are thus placed near a base of legs 228, where for a same degree of bending of legs 228, there is less spatial motion, so side walls 212 can extend out a smaller distance relative to the rest of delivery system 100. Optionally, sidewalls 212 angle out (e.g., so that the ends further from bars 254 are further apart than the ends attached to extenders 224), to define a generally cone shape for each one of bars 254, to assist in such herding.

As can be appreciated, device 200 could be constructed in various ways to allow selective release and engagement of bars 254. In the particular embodiment shown, each bar 254 and side walls 212 associated with the bar are formed of one continuous wire. In the illustrated embodiment, a distal portion of the wire is formed into a post 210, which is maintained in a slot 222, formed in device base 226. Slots 222 optionally prevent twisting or other misalignment of bars 254. In an exemplary embodiment of the invention, a bend 232 is formed in the wire, so that if the wire is pulled back, contact of more distal, parts of the wire with base 226 will cause the wire to pull away from base 226.

In an exemplary embodiment of the invention, a bar controller 230 is provided which is coupled to posts 210 and is selectively locatable at multiple axial location relative to delivery system 100. Retraction of controller 230, will retract the wire and cause bars 254 to open. In this case, bars 254 move in a direction including both axial and trans-axial components. In an alternative embodiment, a distal portion 234 of the wire is curved away from the more proximal portions, so that if the wire is pulled back over a rim of base 226 or through one or more holes defined in it and having a diameter greater than at a proximal portion of slots 222, bars 254 are moved apart. Other methods may be used as well. For example, the wires may act as levers with a part of base 226 serving as a pivot. Pushing the wires together on one side of the pivot, for example by sliding a restraining controller 230 over them, will move the other ends of the wires apart.

Optionally, bars 254 are configured to prevent over compacting. For example, extenders 224 may block each other when they meet. Alternatively, bars 254 may be aligned so that they block each other from further motion once they contact, and the degree of compacting is limited by the degree of recessing of bars 254 relative to extenders 224.

It should be noted that a device similar to device 200 may be constructed from plastic, for example as a solid, rather than as wires, for some embodiments of the invention. However, wires typically have the advantage of being visually unobtrusive.

Splitting Cap Compacting Device

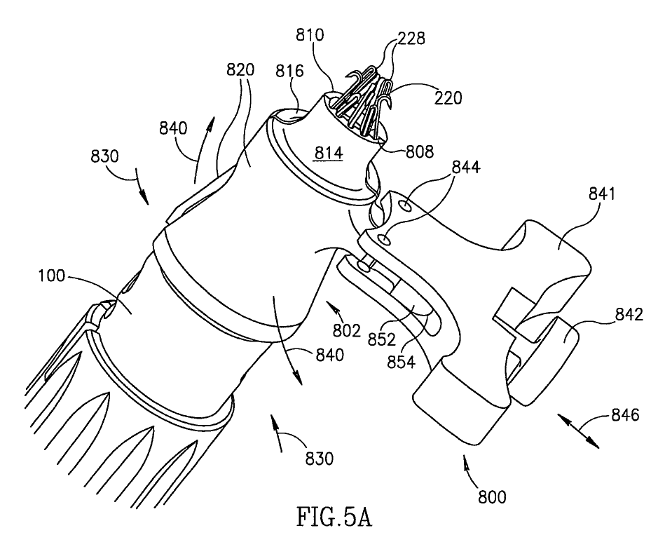

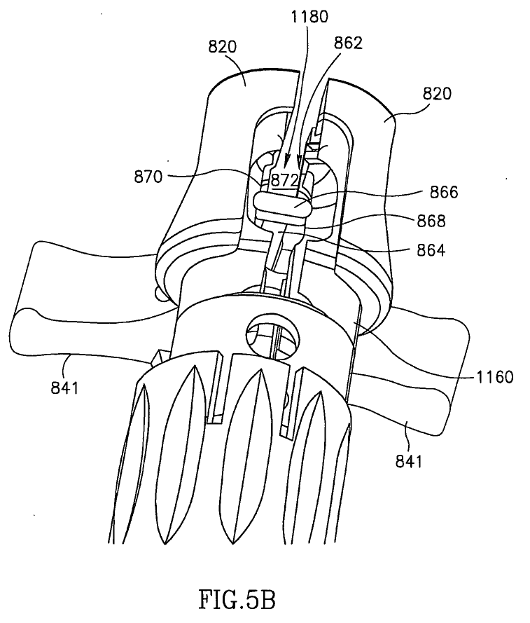

FIGS. 4 and 5A are isometric and schematic views of a splitable leg compacting and arranging cap 800 and its operation, in accordance with an exemplary embodiment of the invention. Cap 800 comprises a body 802 which terminates in leg holding lips 808 and 810. In an exemplary embodiment of the invention, cap 800 is adapted to be split so that lips 808 and 810 move apart, releasing legs to an expanded configuration. In an exemplary embodiment of the invention, body 802 is formed of two halves 820 and a handle 841, which can be manipulated to selectively split cap 800. Optionally, cap 800 can also be selectively closed, to compact legs 228.

In an exemplary embodiment of the invention, halves 820 are adapted to be mounted on delivery system, 100, for example by an internal ridge 806 engaging a matching slot or ridge in delivery system 100. Other mounting methods can be used. When body 802 is split, halves 820 move in directions 840, thereby releasing arranging cap 800 from delivery system 100. In an exemplary embodiment of the invention, halves 820 define at their proximal ends cone shaped sections 814 and 816, which terminate at lips 808 and 810. Optionally, each of lips 808 and 810 defines a plurality of notches 822, each of which notch can hold one of legs 228.

In an exemplary embodiment of the invention, handle 841 includes a push-button 842, however, other control types can be used. When button 842 is pushed in a direction 846, halves 820 move apart in directions 840. Conversely, when button 842 is released, halves 820 move in directions 830, to close. Button 842 and the splitting action are optionally spring loaded, for example so body 820 remains normally unsplit.

Various splitting mechanism may be used for splitting apart cap 800. In one example, each of halves 820 includes an extension 852 that fits inside handle 841. These extensions are crossed, like scissors. Thus, advancing button 842, forces the extensions apart and the cap splits. In an alternative embodiment, pins or projections 844 serve as hinges for extensions 852 which do not cross, and splitting is achieved by button 842 engaging a rounded section 854 of extensions 852, on their outside (e.g., with an arc section), so that extensions 852 move together, thereby splitting cap 800.

While cap 800 is shown to be pre-split along its entire length, this is not the case in all embodiments. For example, the cap my be a single piece adapted to split along the slit shown, under stress. Alternatively or additionally, the split area between body 802 and handle 841 is not completely split and serves as a hinge. It should be noted that depending on the location of the pivot point for the splitting and the splitting mechanism used, halves 820 can move apart in various ways, including different angles being formed between the halves and substantially parallel separation of halves 820. The type of relative motion of the halves may be an issue in embodiments as described below, where a selective release of legs from either side of the cap is desired to be synchronized in a certain manner. In some embodiments, cap 800 can be opened and closed several times. In other, opening damages the cap so reclosing is not feasible.

In an exemplary embodiment of the invention, cap 800 is provided pre-mounted on delivery system 100 and is used during eversion and released only after insertion into the body. Alternatively, cap 800 may be release before insertion and optionally a different compacting method used for insertion. Alternatively or additionally, cap 800 may be mounted by a physician, for example before or after eversion. In this case, a forceps may be used to guide legs 228 into notches 822. Optionally, a leg manipulation tool, for example as described below, is used to guide or move legs 228.

Optionally, cap 800 is used to assess the size of the graft being used. When cap 800 is split, legs 228 expand outwards until the radius of the graft is reached. At this point, further expansion is reduced or prevent. By slowly splitting cap 800, the radius of the graft can be assessed. Optionally, suitable markings are provided on handle 841, possibly with through holes for viewing the position of extensions 852. This method of graft measurement may also be used in other leg compacting and releasing embodiments described herein. Optionally, such graft measurement is used to assess if a correct connector and/or graft vessel are being used and/or to help in determining a size of incision to make in a target vessel.

In an exemplary embodiment of the invention, the connector used is a medallion type connector, for example as described in WO 03/026475, the disclosure of which is incorporated herein by reference. In such a connector, each leg has an associated medallion which may migrate along the leg. In an exemplary embodiment of the invention, lips 808 and 810 prevent the medallions from passing by them along legs 228.

A potential benefit of arranging legs in this and other embodiments is for correct eversion. Another potential advantage is for inserting into a blood vessel, where arranging of the legs can serve to ensure a proper spacing between the legs. As described below, a leg manipulator may be used to move legs if they re not correctly positioned.

Split Graft Capsule

FIG. 5B illustrates another use of a splitable cap 800, which may be instead or in addition to leg arrangement. In ring-type connectors, a graft is passed through a side of the delivery system, through an opening in the ring of the connector and out between the legs. This path may require using a special snare. In an exemplary embodiment of the invention, it is noted that a connector may include a split in the ring or may be formed of a plurality of individual clip-like elements that act together when deployed as a single connector. In an exemplary embodiment of the invention, a capsule 1160 is splitable on either side, lengthwise. Cap 800 holds the capsule and when it is split, using knob 842 (not visible) capsule 1160 is split as well. In the embodiment illustrated, capsule 1160 is actively split. For example, an extension 864 of control 842 may terminate with an non-circular end 866, which is placed in a far (relative to picture plane) slot 872 of capsule 1160. When control 842 is rotated and end 866 apply forces to sides 868 and 870 of slot 872, forcing capsule 1160 open, optionally thereby also opening cap 800. Optionally, capsule 1160 does not fall apart, as cap 800 is holding it. The graft can then be placed in a slot 1180 and then capsule 1160 is closed. Optionally, once mounted on the delivery system, the delivery system prevents the capsule from splitting. In an alternative embodiment, capsule 1160 is provided split and then assembled around the graft, without cap 800. Alternatively to cap 800, other capsule opening and/or restraining devices may be used, including for example, a rubber band to keep capsule 1160 in one piece while being opened by hand or with forceps.

A reference 862 shows an approximate mounting location for the connector segments.

Leg Arranging Notches

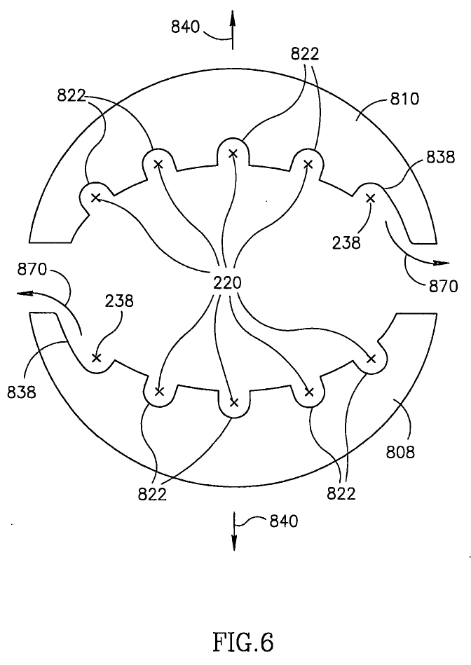

FIG. 6 is a top view of lips 808 and 810, showing the position of notches 822, in accordance with an exemplary embodiment of the invention and also showing optional special notches 838 and end legs 238, which will be described below. These lips may be used for arranging legs, for example prior to eversion and/or prior to insertion.

Leg Release Arrangement

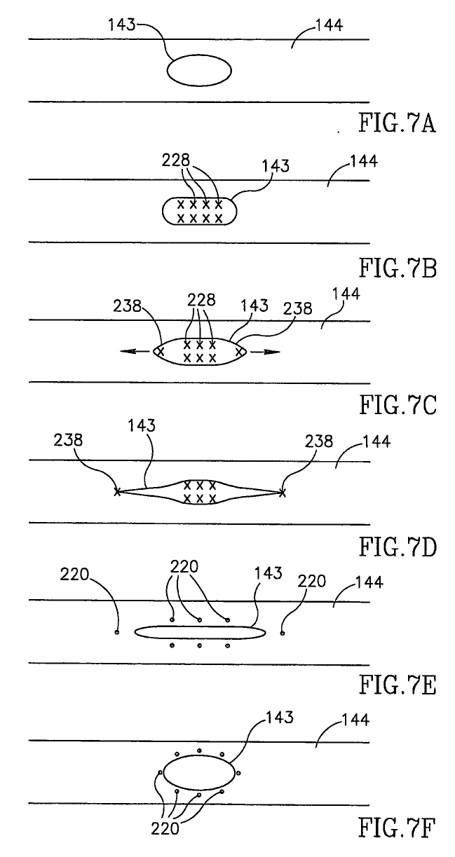

FIGS. 7A-7F are top views of an aperture in a blood vessel demonstrating the positions of anastomotic connector legs as they are selectively released during connection to the host vessel, in accordance with an exemplary embodiment of the invention.

FIG. 7A shows an aperture 143 formed in a target vessel 144. FIG. 7B shows the positions of legs 228 as inserted into the aperture 143. The inventors have found that in some cases the lips of aperture 143 are flaccid and hooks 220 do not all engage lip tissue and/or engage it at a point too close or too far away from the actual edge of the aperture. In an exemplary embodiment of the invention, the connector legs are used to stretch aperture 143 so that its lips are tighter and there is better control over the points where hooks 220 engage. It should be noted that aperture 143, when made by incision is typically thinner. An exemplary incision maker is described in a PCT application filed on same date as this application in the Israel receiving office of the PCT, by a same applicant “By-Pass Inc.”, having the title “BLOOD VESSEL CUTTER” and having attorney docket number 088/03505, the disclosure of which is incorporated herein by reference.

FIG. 7C shows an embodiment where one (or more) end legs 238 each engages an opposite end of aperture 143 and stretches it. In one embodiment of the invention, the legs of the connector are pre-stressed to achieve a layout as shown in FIG. 7C. Alternatively, this configuration is achieved by selective release of legs and/or by positioning of legs using a leg compacting device.

FIG. 6 shows how special notches 838 can be defined which release legs 238 early, before legs can escape notches 822. Optionally, the notches are positioned so that the legs touch near hooks 220 when held by the notches in a closed position of cap 800. While notches 838 are shown to be symmetrical, if the splitting of cap 800 is around a pivot, an asymmetrical design may be useful for embodiments where substantially simultaneous release of legs at opposing sides is desired. Alternatively, an intentional earlier release of one of legs 838 before the other one of legs 238, is practiced.

FIG. 7D shows the further stretching of aperture 143 by legs 238, at which point legs 228 may be released and then retracted. Optionally, the leg compacting device is provided with a tactile feedback so that a user can feel when the device has reached a second position, so the user can pause after releasing legs 238 and before releasing legs 228. In an exemplary embodiment of the invention, an incision is between 2 mm and 3.5 mm prior to stretching and is stretched to between 3 and 4.5 mm or 5 mm.

FIG. 7E shows the final position of hooks 220.

FIG. 7F shows the resulting connection profile after the delivery system is removed, in which the natural elasticity of the vessel and/or design of the connector shapes the final profile. It should be noted that a greater or smaller number of legs may be used than the 8 shown, depending, for example, on the size of the connection. This method may also be used for non-linear and non-circular holes. In particular, three legs 238 may be used for dealing with a triangular aperture. In addition, the positioning of legs 238 may depend not only on the shape and orientation of aperture 143 but also on the graft vessel, for example, it may be desirable to have legs 238 positioned at the points of greatest and smallest angle between the graft and target vessels.

It should be noted in this and other embodiments, that the connector need not be formed of one continuous piece, especially after deployment. For example, the connector may be a set of staples that are delivered together and are possibly attached during deployment.

Leg Release Design

Alternatively or additionally to providing cap which releases legs 228 and 238 in a desired order, in an exemplary embodiment of the invention, the connector is pre-configured such that the end legs extend radially more than the inner legs. Alternatively or additionally, the delivery system aims the legs (e.g., by angling the apertures and/or medallions where they exit the delivery system) more to the sides. Thus, FIG. 7B shows the legs when confined by a noose. FIG. 7C shows partial release and FIG. 7D shows complete release. Closing the anastomosis allows the graft vessel to affect the configuration so FIGS. 7E and then 7F are achieved.

In a ring type or two part connector, the connector or the legs parts can be configured to provided extended and/or stronger radial motion for only some of the legs. In multi-part connectors that comprise a plurality of unconnected legs, the side legs may be selected to be more bent our and/or stronger. In an exemplary embodiment of the invention, the legs used to stretch the incision are two legs from either side of the connection (e.g., when arranged along the incision as in FIG. 7B). In one example the two upper right legs extend to the right and the two bottom left legs extend to the left.

Tear Over-Tube

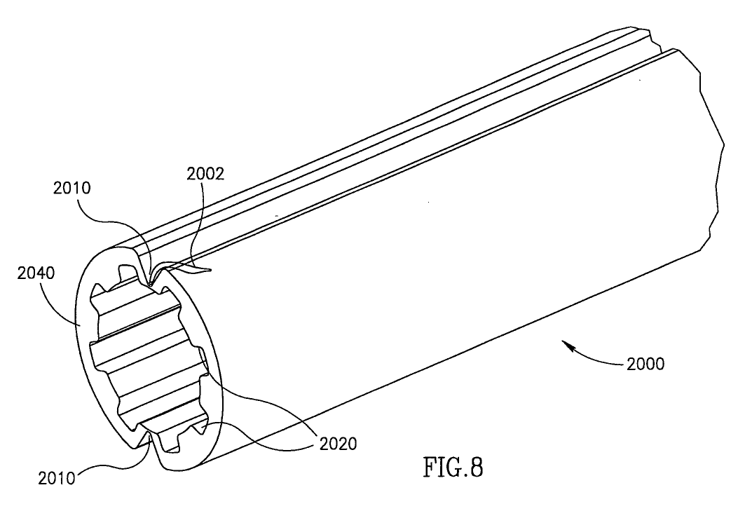

FIG. 8 is an isometric view of a compacting overtube 2000, in accordance with an exemplary embodiment of the invention. Overtube 2000 is an elongate tube-like object having a diameter suitable for a desired leg compacting geometry. Optional, tube 2000 defines a plurality of slots 2020 in its interior, each of the slots adapted to engage a single connector leg 228.

In an exemplary embodiment of the invention, overtube 2000 has a uniform diameter. Alternatively, tube 2000 is tapering, on its inside at least, for example to conform to a cone shape of legs 228.

In an exemplary embodiment of the invention, a notch 2010 or other weakening of over tube 2000 is provided along the over tube. In an exemplary embodiment of the invention, overtube 2000 is removed by tearing or cutting at the notch, as described below.

Optionally, overtube 2000 is made of a material which can be heat-shrunk onto the legs and/or delivery system.

In an exemplary embodiment of the invention, each notch 2020 holds one leg 228 and hooks 220 are outside of overtube 2000, for example, curved over a front face 2040 of the overtube. Optionally, slots (not shown) are formed in face 2040, to receive hooks 220. Alternatively, hooks 220 are straightened by overtube 2000. Alternatively or additionally, hooks 220 are received in notches 2020, further compressing legs 228 together. If heat-shrunk, the shrinking can be uneven on the legs and/or the delivery system.

In an exemplary embodiment of the invention, notch 2010 is torn using a tear tab 2002. In one example, tab 2002 comprises a wire that runs along notch 2010 (e.g., is embedded therein) and extends out as visible tab 2002. Pulling on tab 2002, tears the notch. In another example, the bottom of notch 2010 includes two parallel slits, and tab 2002 is attached to the material between the two slits. Alternatively, a knife is used to cut notch 2010, for example, manually, or a knife mounted on the delivery stream. Alternatively, delivery system 100 is splitable and as it splits, the delivery system also splits apart overtube 2000. Alternatively, a cone shape object having a larger base diameter than overtube 2000 is advanced over legs 228, from the direction of delivery system 1000, splitting overtube 2000.

In an exemplary embodiment of the invention, overtube 2000 is provided mounted on legs 228, when delivery system 100 is packaged. A snare, described for example in a PCT application titled “SNARE”, filed by a same applicant “By-Pass inc.” in the Israel receiving office of the PCT on same date as this application, and having attorney docket number 088/03612, the disclosure of which is incorporated herein by reference, is optionally used to convey a graft through delivery system 100. Slight retraction of overtube 2000 may reveal hooks 220 and allow the graft vessel to be everted over them. Optionally, overtube 2000 is cut after hooks 220 are inserted into a target vessel.

In an alternative use, legs 228 are inserted, hooks first, into overtube 2000, which is then optionally cut to form a short ring.

Radial Leg Compacting

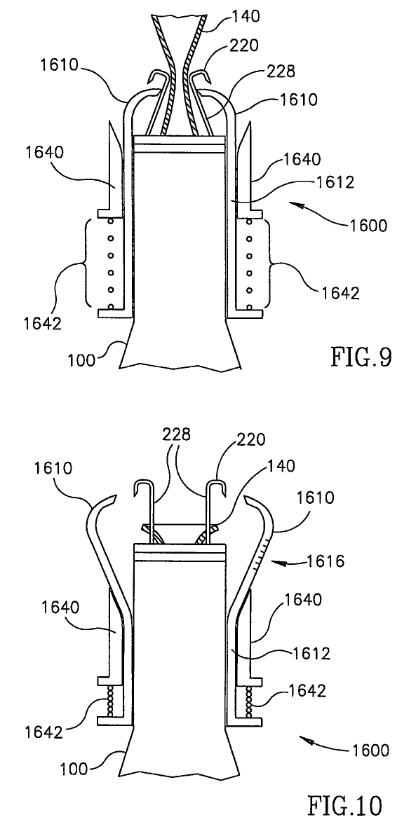

FIGS. 9-12 are schematic cross-sectional, side and isometric views of a radial leg compacting and arranging device 1600, in accordance with an exemplary embodiment of the invention.

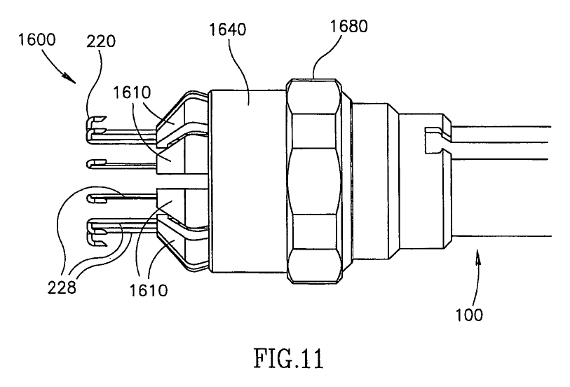

FIGS. 9 and 10 are side cross-sectional view of device 1600, shown mounted on a delivery system 100. While not shown, an optional locking mechanism is provided, to prevent inadvertent axial motion of device 1600. For example, a slot and pin arrangement or a friction arrangement may be used for preventing axial motion.

FIG. 9 shows device 1600 is a resting position (for a normally closed-type device; normally open devices are also possible), in which each of legs 228 is engaged by a finger 1610. Optionally, fingers 1610 are extensions of a body 1612, which fingers are maintained pointed radially inwards by an outer sleeve 1640. A spring 1642 is optionally provided to maintain outer sleeve 1640 in its resting position.

In FIG. 10, sleeve 1640 is retracted, allowing fingers 1610 to spread out, releasing legs 228. As shown, graft 140 is mounted in the configuration of FIG. 9 and after eversion is completed, legs 228 are released.

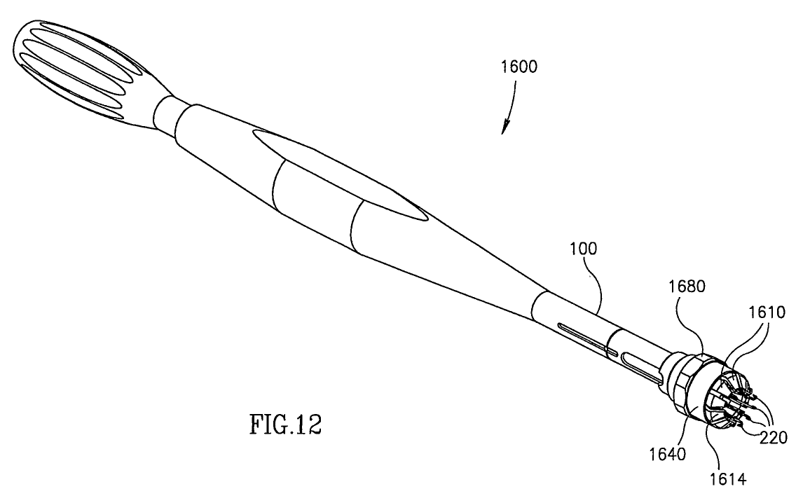

FIG. 11 is a side view of a variation of device 1600, in which sleeve 1640 is controlled by a rotating ring 1680, whose rotation advances and retracts sleeve 1640. Alternatively or additionally, the rotation advances and retracts fingers 1610.

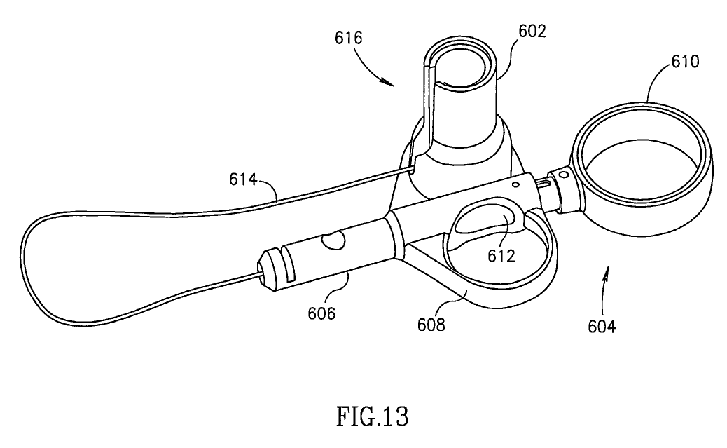

FIG. 12 is an isometric view of device 1600 mounted on delivery system 100. It should be noted that fingers 1610 are shown to form a complete ring, when compressed together. Alternatively, they do not touch. Optionally, a notch 1614 is defined at the end of each finger 1610, to act as a receptacle for a leg 228.

Optionally, a plurality of markers are formed along fingers 1610, at locations 1616 (FIG. 10). If sleeve 1640 is retracted slowly, legs 228 stop expanding at the point where the inner radius defined by fingers 1610 is approximately equal to the radius of the graft. The markings indicate the radius of the graft.

In an exemplary embodiment of the invention, device 1600 is provided on a packaged delivery system 100. Alternatively, it may be added after the fact by a user.

Lasso System

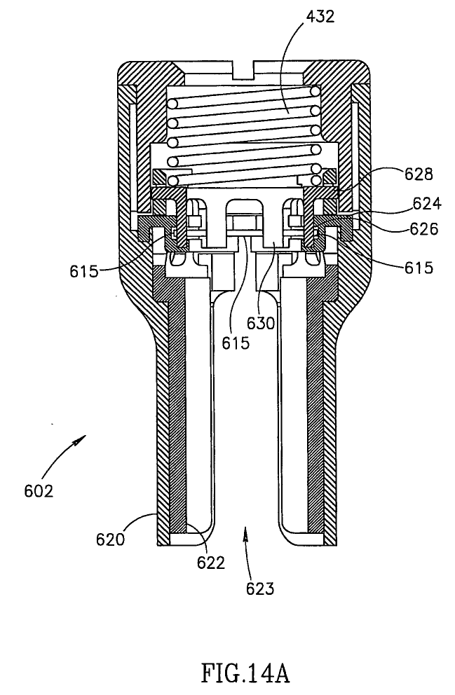

FIG. 13 is an isometric view of a lasso-based leg arranging device and controller system 600, in accordance with an exemplary embodiment of the invention. A lasso noose 615 (not shown) is used to compact legs 228. To assist in mounting noose 615 on legs 228, a leg arranging device 602 is provided, which allows a noose to close only once legs 228 are properly positioned with respect to noose 615. In an exemplary embodiment of the invention, a controller 606 is provided for selectively tightening and releasing noose 615 and, optionally, for tearing the lasso.

Controller 604 comprises a body 606 and a plunger 610, although other designs are possible. In an exemplary embodiment of the invention, controller 606 contains an internal spring (not shown) configured to push out plunger 610. Noose 615 optionally comprise a wire 642 (not shown) or string folded in half (the folded area defining a noose section) and optionally covered by a protective tube 614. Tube 614 is attached to body 606, while the wire is attached to plunger 610. Thus, outward motion of plunger 610, will tend to pull the wire into the tube to tighten the noose. Optionally plunger 610 is locked to body 606, for example, to prevent forces on noose 615 during storage. In an exemplary embodiment of the invention, body 606 contains one or more handles 608 which also have a geometry suitable for holding arranging device 602. In an exemplary embodiment of the invention, a recess 612 is defined in an extension of plunger 610, such that arranging device 602, when placed in handle 608, locks plunger 610.

Optionally arranging device 602 is provided pre-loaded with noose 615. Protective tube 614 is shown entering a slot 616 formed in side of arranging device 602, for this purpose and for receiving a graft vessel.

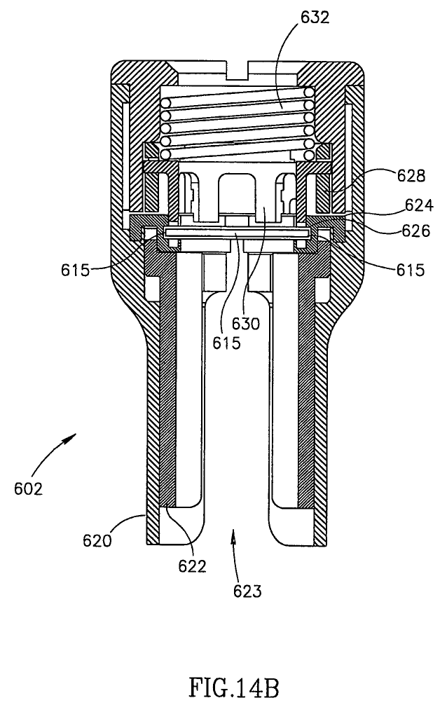

FIGS. 14A and 14B are side cross-sectional views of noose arranging device 602 in two operating states, in accordance with an exemplary embodiment of the invention, in which noose section 615 of the lasso is shown, but tube 614 is not.

Arranging device 602 comprises a body 620 with an inner sleeve 622, both of which open to an opening 623. When a delivery system is inserted into opening 623, sleeve 622 is pressed up, with results as described below. Attached to and inside body 620 is a noose holding ring 624 which includes an inner receptacle 626 for holding noose 615. Ring 624 is positioned adjacent where legs 228 are expected to reach when a delivery system is inserted into opening 623. Noose 615 is, however, prevented from closing on legs 228, by an inner cage 628 which has a plurality of extensions 630 locking noose 615 inside receptacle 626. A solid design for the cage may be used as well. A spring 632 optionally urges cage 628 to be in a closed state. Alternatively, friction is used to prevent inadvertent motion of cage 628.

FIG. 14A shows cage 628 in its closed state. FIG. 14B shows cage 628 in its open state, caused by sleeve 622 moving cage 628 against spring 632. Noose 615 is shown released, but not yet tightened, for clarity.

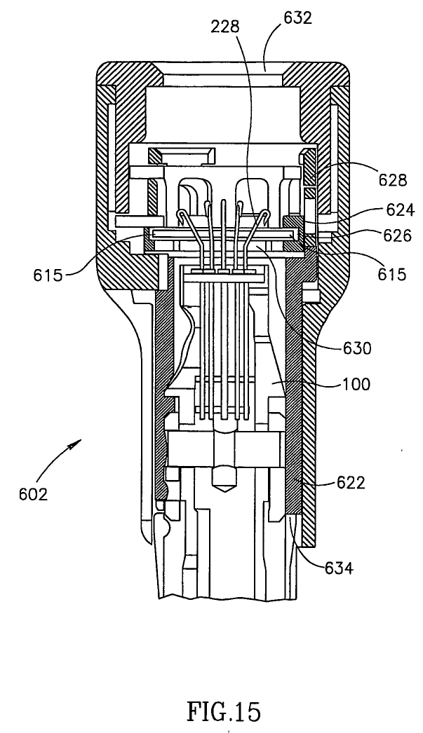

FIG. 15 shows arranging device 602 with a delivery system 100 inserted inside. Delivery system 100 and/or arranging device are adapted to push sleeve 622 by a required amount so that noose 615 will be release at a correct axial position of legs 228. Once noose 615 is released, plunger 610 will pull out and the user can remove delivery system 100 from arranging device 602. Optionally, noose 615 is released adjacent a point on legs 228 other than its final position. However, due to its being tensed and legs 628 being compacted into a cone shape, noose 615 can slide to a final resting position, and further tightening.

At this point, legs 228 are compacted and a graft can be everted on the legs or the legs can be inserted into a blood vessel.

A particular feature of controller 604 in some embodiments of the invention, is that by manipulating plunger 610, the noose 615 of the lasso can be selectively tightened and released. For example, after insertion into a blood vessel aperture, if releasing the legs does not result in a correct leg placement, the noose can be tightened (with the legs being compacted) and a new attempt made. Optionally, controller 604 can be mounted on delivery system 100. Alternatively or additionally, tube 614 is long enough to allow controller 604 to lie comfortably outside the patients body.

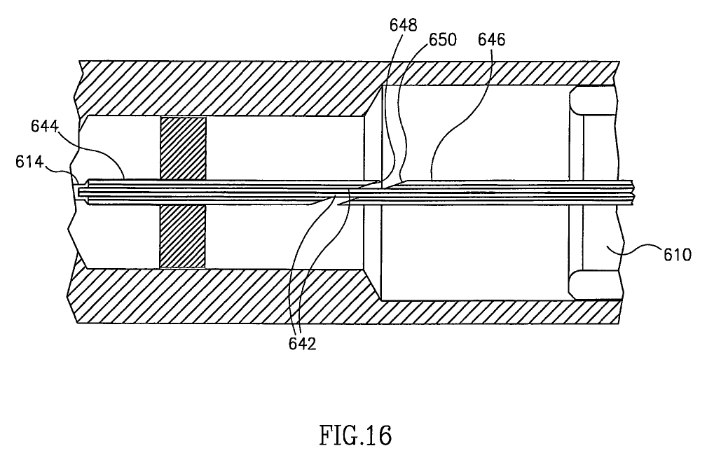

FIG. 16 is a cross-sectional view of a section of a lasso controller, showing a lasso tearing mechanism 640, in accordance with an exemplary embodiment of the invention. Once the use of noose 615 is completed, noose 615 is optionally removed by tearing the lasso wire (642). FIG. 16 shows one possible tearing mechanism, in which protective tube 614 is coupled to a metal tube 644 having an inclined edge 648 and plunger 610 is coupled to a second metal tube 464, having an inclined edge 650. Wire 642 lies within the tubes. When plunger 610 is pushed in all the way, inclined surfaces 648 and 650 contact and then slip by each other, for example, only tube 646 having non-axial motion. The inclined surfaces act as scissors to shear-cut wire 642. Wire 646 is thus detached from the controller and can be removed from the anastomosis area buy pulling on one end thereof. In an alternative embodiment, rotation of plunger 610 relative to body 606 tears the wires. Alternatively, other tearing mechanism, including a hand-held scalpel, may be used. In some embodiments of the invention, wire 642 is only cut at one location, so that it remains attached to controller 604 at all times.

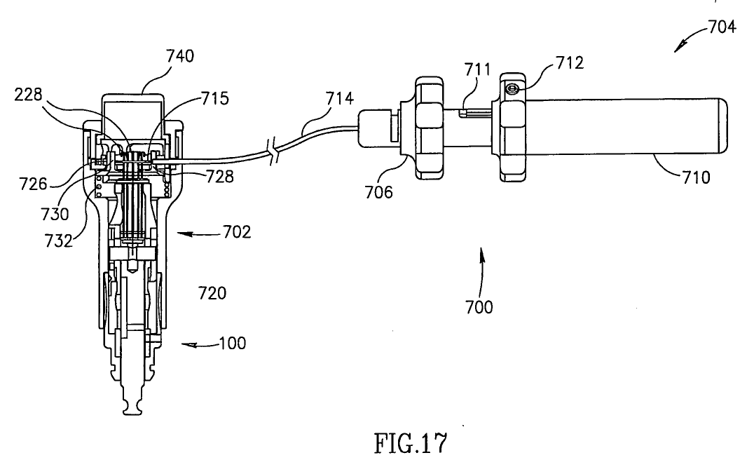

FIG. 17 is a partial cross-sectional view of an alternative embodiment of a lasso arranging system 700, in accordance with an exemplary embodiment of the invention. The reference numbers generally correspond to the reference numbers of FIGS. 13-15 and will not be described again. One possible alteration is in the design of a controller 704, in which locking is achieved by a pin 712 engaging a notch 711, in a body 706 of controller 704. Rotation of a handle 710 releases pin 712 from the notch. Another possible alteration is that instead of automatic release of noose 615 when delivery system 100 is completely inserted, a button 740 is depressed to move a cage 728 out of the way, against an optional spring 732.

While noose 615 has been generally described as being used for compacting legs prior to insertion, it should be noted that it can also be used for an aid for eversion. In an exemplary embodiment of the invention, controller 600 has two retracting positions for plunger 610. One in which the legs are completely compacted together, without leaving a space and one in which a space to pass a graft is provided.

In an alternative use of a leg arranging and/or compacting device, an end-to-end connection of two vessels is supported. The lasso or other leg compactor is used to compact the legs of a connector mounted on a first vessel. The compacted legs are inserted into an end of a second vessel. The lips of the second vessel are mounted on the hooks and then the legs are release. Possibly, the legs are released prior to mounting, until the legs reach the diameter of the other vessel. After mounting on the other vessel, the connection is completed, for example, by pulling and tearing in a medallion type connector.

Pistol Grip Delivery System

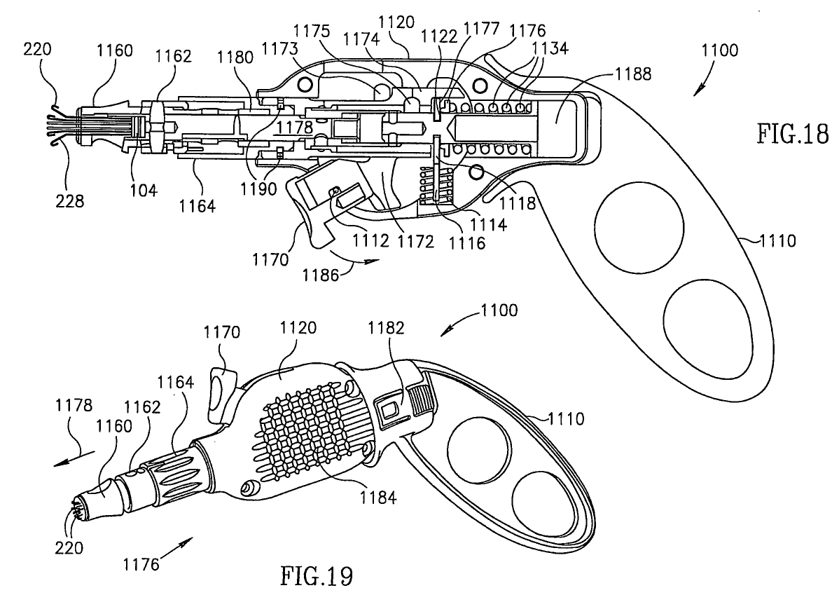

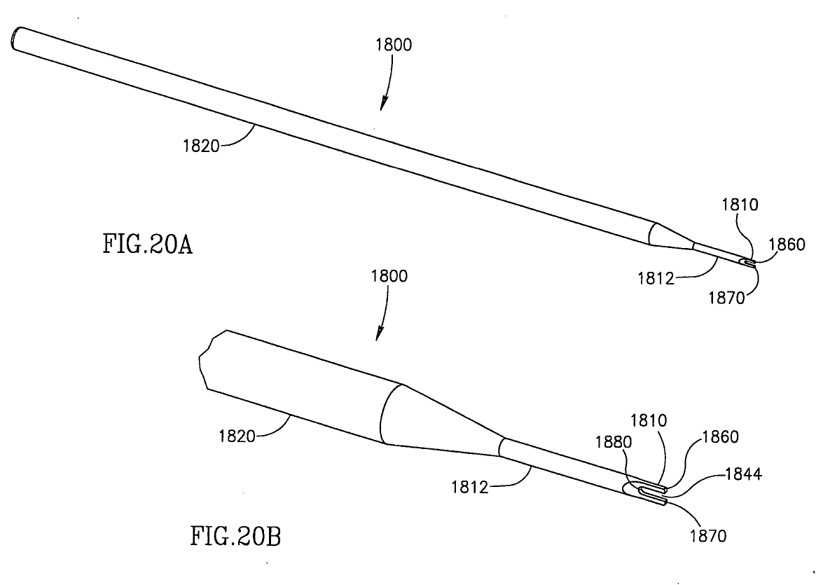

FIG. 18 is a side cross-sectional view of a pistol-grip connector delivery system 1100, in accordance with an exemplary embodiment of the invention. FIG. 19 is an isometric view of pistol-grip connector delivery system 1100. Delivery system 1100 is used to show two features, which are not necessarily both present. A first feature is a pistol grip 1110, which can be optionally attachable at multiple orientations, for example as can be seen by comparing FIG. 18 and FIG. 19, where the grip is rotated 180 degrees. Other optional features which may assist in gripping and using of delivery system 1100, are a roughed area 1184 and a trigger 1170 used to activate delivery system 1100.

A second feature of delivery system 1100 which may be provided alternatively or additionally to the pistol like features is a controlled deployment of connector 104, in which a user is not required to move the delivery system and/or in which power for tearing is provided by a spring released by a user, rather than directly by the user.

System 1100 comprises a body 1120 on which pistol grip 1110 is attached, for example using a snap attachment 1182. In an exemplary embodiment of the invention, snap attachment 1182 allows positioning and/or attaching of grip 1110 at multiple positions, for example, 2, 6, 8, 10 or a smaller, intermediate or larger number of positions, for example, every 20, 30 or 45 degrees. A trigger 1170 is configured, in this embodiment to pivot, and follow a path 1186. A safety pin 1112 is optionally provided to prevent motion of the trigger unless also pressed in a small amount.

In an exemplary embodiment of the invention, system 1100 operates in two stages. In a first stage, trigger 1170 directly moves hooks 220 relative to a tip 1160. In the example shown, tip 1160 is advanced, while legs 228 do not move. Alternatively, legs 228 can be retracted, while tip 1160 remains in place. In either case, optionally, a user is not required to move body 1120 relative to the patient. A potential advantage of not actually moving legs 228 is that retraction of the legs may cause them to tear tissue and/or distort.

In a second stage, a spring 1134 is released to tear legs 228 off of the delivery system, again optionally without moving body 1120. In an exemplary embodiment of the invention, the following mechanism is used. Connector 104 is attached to a piston 1178, by a coupler 1162, which piston is coupled to a compressed spring 1134. However, a plate 1177 lies in a notch 1122 of piston 1178 and the other end of spring 1134 to piston 1178. In the embodiment shown, a ring 1176, which is optionally fixed to body 1120 also serves as a base for spring 1134, and which ring rests against plate 1177. In an exemplary embodiment of the invention, plate 1177 is part of a rectangular element 1116 with an aperture 1118 which is larger than a cross-section of piston 1178 and through which piston 1178 fits. A spring 1114 urges element 1116 in a direction which would release plate 1177 from notch 1122, however, an extension 1174 prevents this motion. Extension 1174 is optionally coupled to a tube 1180 on which a barrel 1164 and tip 1160 are mounted. The first stage advances tube 1180, and also extension 1174. Sufficient advancement clears the path for plate 1177, which is urged to unlock piston 1178 by spring 1114. Spring 1134 expands, pulling back connector 104, while tip 1160 does not move, thereby tearing off legs 228 and completing the anastomosis. Optionally, plate 1177 in its unlocking position prevents extension 1174 and thus tip 1160 from retracting under the force of spring 1134. Alternatively or additionally, one or more pins 1190 pop out once they are advanced with tube 1180 past the edge of body 1120. Once they pop out, tube 1180 can no longer be retracted. Alternatively or additionally, an extension 1175 of trigger 1170 locks tube 1180 to body 1120. In an exemplary embodiment of the invention, an extension 1173 of a body 1172 of trigger 1170 is what advances tube 1180 trigger

In an alternative mechanism, trigger 1170 itself moves plate 1177 out of the way, for example using a pin (not shown) on extension 1175.

Optionally, a sponge 1188 or an oil cylinder is provided to slow the expansion of spring 1134, thereby preventing delivery system 1100 from jumping. Alternatively or additionally, sponge 1188 does not slow down the spring opening. Instead, it serves to reduce a recoil effect when the spring hits body 120. Reducing recoil may prevent the user from moving handle 1110.

Alternatively, a battery powered motor may be used for the tearing. It should be noted that an exemplary force required to tear legs 228 is about 25 Kg. However, the triggering mechanism allows the user to apply a small force, and, optionally, not be subject to jumping of device and/or not be required to move the device.

Optionally, a safety (not shown) is provided which prevents trigger 1170 from activating spring 1134 if a capsule with a connector is not placed into barrel 1180.

Legs 228 may be compacted using the methods described above. Alternatively, for this and other types of delivery systems, the legs may be pre-stressed to be bent inwards. This helps eversion. Once a graft is mounted on the legs, the graft tends to pull the legs apart, so a lasso or other mechanism is used for compacting. It should be noted that in this case the legs may be shorter and/or the delivery system is not required to selectively advance and retract the legs.

In an alternative solution, also useful for multiple types of delivery systems, the connector legs are pre-stressed to bend out or be straight. A band, for example a ring of silicon, or a string is provide don the connector in the packaging, to compact the legs. After eversion, this band is cut, and removed. optionally, a string is attached to the band, to prevent it from getting lost after removal.

Leg Manipulator

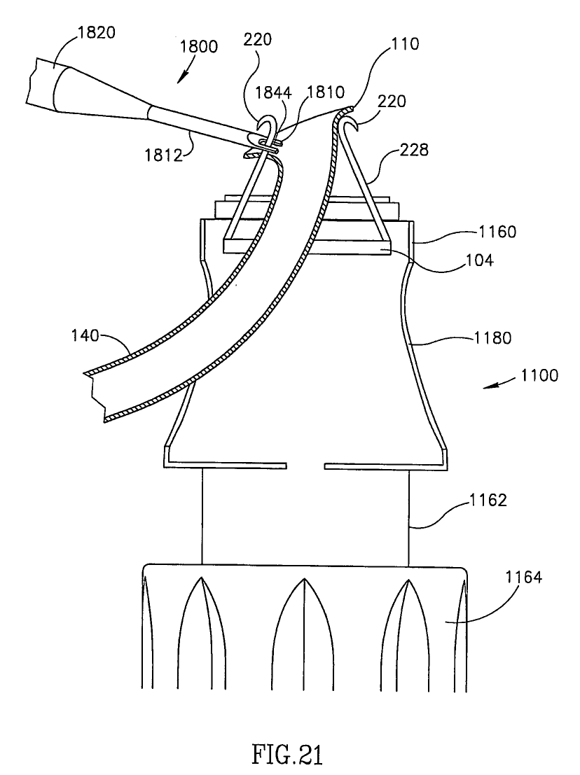

FIGS. 20A and 20B are isometric views of a leg manipulator 1800, in accordance with an exemplary embodiment of the invention. Manipulator 1800 comprises a handle 1820 having an optional taper section 1812 from which a tip 1810 extends, optionally at an angle. Alternatively or additionally, section 1812 may be at an angle to shaft 1820. In an exemplary embodiment of the invention, tip 1810 comprises twin prongs 1860 and 1870, which optionally meet at a curved section 1880. Thus, tip 1810 defines a leg receptacle 1844, which is adapted for partially encircling an anastomotic connector leg 228. In an exemplary embodiment of the invention, prongs 1860 and 1870 are in on plane and extend about two or three times the width of a leg 228. For example, a leg may have a width of between 0.2 and 0.35 mm and a thickness of between 0.1 and 0.2 mm. As will be described below, manipulator 1800 is used for moving individual legs 228. Optionally, prongs 1860 and 1870 are made thin enough so that a leg can be manipulated without affecting nearby legs.

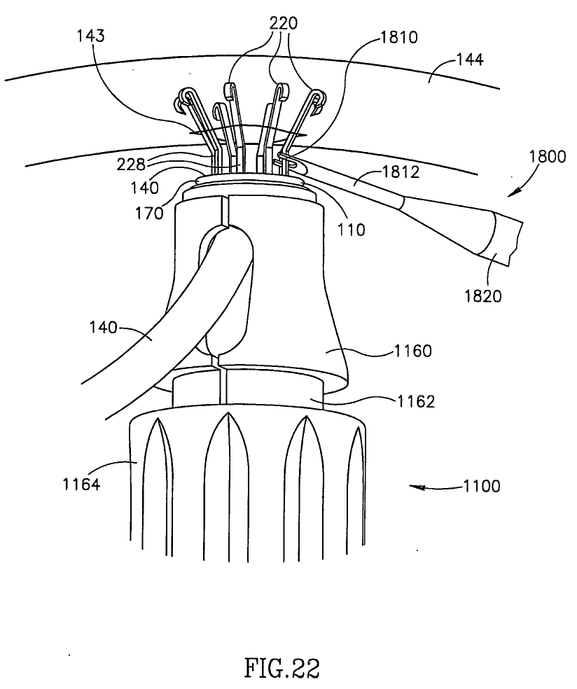

FIG. 21 is a partially cross-section side view of a delivery system 1100 showing leg manipulator 1800 assisting in eversion and/or movement of a graft 140 over a leg 228. In one example, manipulator 1800 is used as an eversion assistance device, such as shown in WO 03/026475, the disclosure of which is incorporated herein by reference, for example in FIG. 6. Alternatively, for example with reference to FIG. 4, manipulator 1800 can be used prior to eversion, for example to arrange the legs or to assist in compacting, for example by pushing legs to individual notches 822, if they do not align correctly by themselves. Alternatively or additionally, manipulator 1800 is used to untangle leg tangles.

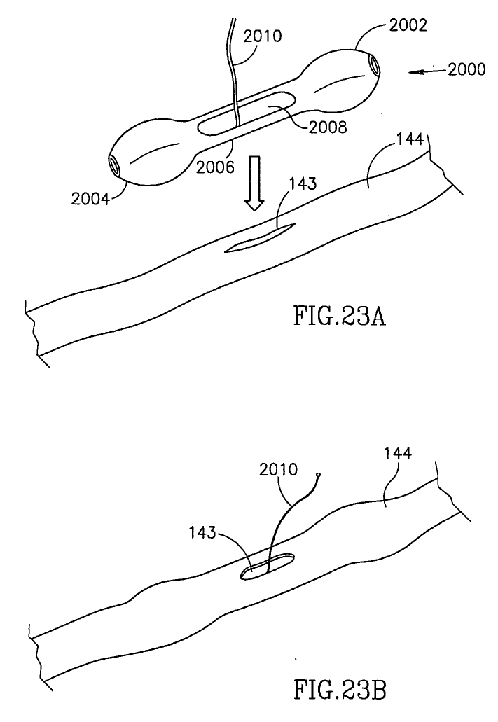

FIG. 22 is a side view of leg manipulator 1800, demonstrating leg manipulation during insertion of legs 228 into an aperture 143 of target vessel 144, in accordance with an exemplary embodiment of the invention. when legs 228 are released or if other insertion methods are used, one or more of legs 228 may pop out of aperture 143. Manipulator 1800 is optionally used to push such legs 228 back into aperture 143. Alternatively or additionally, manipulator 1800 is used to arrange legs 228 and especially hooks 220 in a desired alignment with respect to aperture 143. Alternatively or additionally, manipulator 1800 is used to assist a hook 220 in piercing the wall of target vessel 140.

Leg Manipulator Specifications

In an exemplary embodiment, receptacle 1844 may be 0.65 millimeters in diameter (e.g., width), for example when hook 220 has a diameter of 0.6 millimeters. Alternatively or additionally, receptacle 1844 may be as large as 0.70 millimeters or larger or as small as 0.61 millimeters or smaller. Hook 220 may have a larger diameter, for example when used in anastomosis of large vessels, the size of receptacle 1844 will be appropriately changed.

In an exemplary embodiment, each prong 1860 and 1870 of tip 1810 is sized to fit between two adjacent connector legs 228 and may have a cross-section side-to-side size of 0.45 millimeters, when the space between legs 228 is, for example 0.40 millimeters. Alternatively or additionally, prongs 1860 and/or 1870 may have a sided-to-side size as large as 0.50 or more millimeters or as small as 0.41 millimeters or less.

In an exemplary embodiment, receptacle 1844 is shown with “U” shape 1880, in which prongs 1860 and 1870 are parallel to each other and spaced apart. In other exemplary embodiments, prongs 1860 and 1870 may be angled to each other and/or there may be a V shaped meeting. Alternatively or additionally, the prongs may lie on different planes from each other and/or different from neck 1812. The shapes of legs 228 may for example, influence the difference in shape.

Shunt

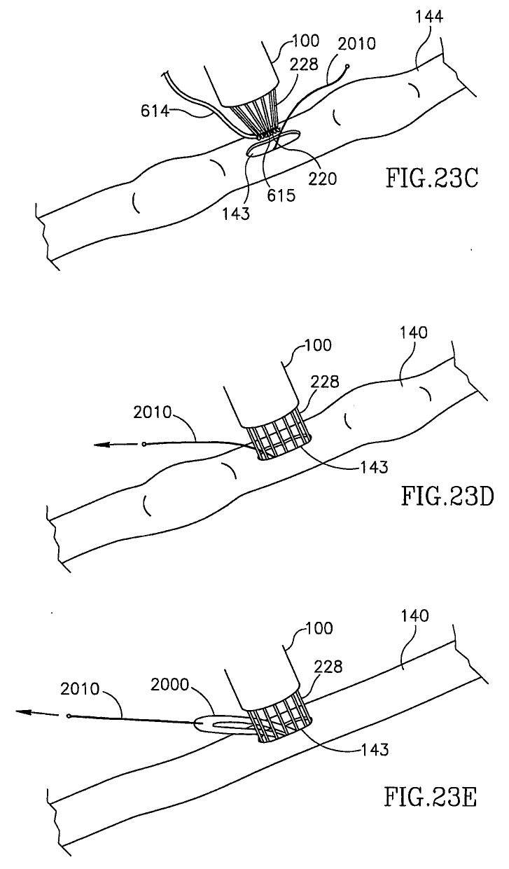

FIGS. 23A-23E illustrate the use of a shunt 2000, in accordance with an exemplary embodiment of the invention. In some embodiments of the invention, the anastomosis process may take more than a few seconds, for example one minute, two minutes or more. During this time, aperture 143 may be open. In some cases, it is not practical to stop the flow of blood in target vessel 144, especially if it is a single coronary artery.

Shunt 2002 can be a standard shunt used to bypass from one side of aperture 143 to the other. For example, the shunt can be formed of an elastic material, such as silicone and have a first side 2002 including an entrance (not shown) and being capable of expanding to a diameter greater than that of the coronary artery, and a second side 2004 with the same properties. An opening 2008 in the middle of the shunt corresponds to the anastomosis area and is bordered by one or more shunt blood passages 2006. An optional removal thread 2010 can be used to pull the shunt out of the blood vessel when the anastomosis is near completion.

FIG. 23A shows shunt 2002 before insertion into target vessel 144 through aperture 143.

FIG. 23B shows the shunt inserted and also shows how sides 2002 and 2004 expand to greater than the vessel diameter, to reduce flow from bypassing the shunt. Removal thread 2010 is visible.

FIG. 22C shows a delivery system being used, with legs 228 being held by a lasso 615.

FIG. 22D shows the lasso having been removed after legs 228 were positioned in a desirable manner. At this point, hooks 220 are optionally retracted so that they engage the wall of vessel 144. Optionally, one of hooks 220 is moved out of the way, for example, using manipulator 1800, to allow room for the shunt between two adjacent legs 228.

FIG. 22E shows shunt 2000 being removed by pulling on removal thread 2010. In an exemplary embodiment of the invention, hooks 220 hold aperture 143 to prevent its tearing by the pulling. In some cases, removal of the shunt requires that legs 228 be able to move apart. One possibility is if legs 228 are long, for example having more than 10, 15, 20 or 30 mm free space between the vessel and the delivery system. Another possibility is if the connector is of a type where the legs each lock to a separate element and the legs are optionally not attached to each other. in this case, the graft, on which the legs are mounted allows for some freedom of motion between the legs, so the shunt can be removed.

An alternative use of the ability of the legs to move is for a side-to-side connection, in which one side vessel is placed between the legs in a trans-axial direction. A protective sheet may be provided to prevent the sides of legs 228 from cutting into the side vessel. In an exemplary embodiment of the invention, the delivery system is split to go on either side of the connection, optionally with a ring provided between the two vessel during the procedure to keep the connection to a correct shape.

Application

The above leg arranging and graft manipulation methods and/or devices may be used with many types of delivery systems. In particular, the following documents, the disclosures of which are incorporated herein by reference describe connectors, delivery systems and/or other tools and methods which are useful in conjunction with embodiments of the prevent invention:
PCT/IL02/00790, filed on Sep. 25, 2002, now published as WO 03/026475;
U.S. Ser. No. 60/492,998 filed on Aug. 7, 2003.
PCT/IL02/00215, filed on Mar. 18, 2002, now published as WO 02/074188;
PCT/IL01/01019, filed on Nov. 4, 2001, now published as WO 02/47532;
PCT/IL01/00903, filed on Sep. 25, 2001 now published as WO 02/30172;
PCT/IL01/00600, filed on Jun. 28, 2001, now published as WO 02/47561;
PCT/IL01/00267, filed on Mar. 20, 2001, now published as WO 01/70091;
PCT/IL01/00266, filed on Mar. 20, 2001, now published as WO 01/70090;
PCT/IL01/00074, filed on Jan. 25, 2001, now published as WO 01/70119;
PCT/IL01/00069, filed on Jan. 24, 2001, now published as WO 01/70118;
PCT/IL00/00611, filed on Sep. 28, 2000, now published as WO 01/41624;
PCT/IL00/00609, filed on Sep. 28, 2000, now published as WO 01/41623,
PCT/IL00/00310, filed on Mar. 20, 2000, now published as WO 00/56228;
PCT/IB00/00302, filed on Mar. 20, 2000, now published as WO 00/56227;
PCT/IL99/00674, filed on Dec. 9, 1999, now published as WO 00/56223;
PCT/IL99/00670, filed on Dec. 8, 1999, now published as WO 00/56226;
PCT/IL99/00285, filed on May 30, 1999, now published as WO 99/62408; and
PCT/IL99/00284, filed on May 30, 1999, now published as WO 99/62415. The disclosure of all of these applications, which designate the US and were filed in English, are incorporated herein by reference.

In addition, two PCT applications filed on same date with the present application, by applicant “By-Pass inc.”, and describing anastomotic connectors are, “Sliding Surgical Clip” and “Anastomotic connectors”, attorney docket numbers 088/03506 and 088/03736, the disclosures of which are incorporated herein by reference. A provisional application filed on even date with the instant application, by applicant Loshakove et al., and having attorney docket number 088/03695 is also incorporated herein by reference and describes an exemplary delivery system.

It should be noted that the term “connector” should be construed broadly to include various types of connectors, including one part, two part and multiple part connectors, some of which when deployed, result in a plurality of individual clip-like sections.

The term “eversion”, where used means not only complete eversion of 180 degrees, but also partial eversion or flaring, for example of 90 degrees. Also, in some embodiments, mounting without eversion is provided.

Measurements are provided to serve only as exemplary measurements for particular cases. For example, 2.5 millimeter diameter of a connector 101 and the size of the aperture 143 may apply to a connector that is connected to a LIMA, while connectors 101 and/or apertures 143 that are used in conjunction with other vessels, for example a vein, may have alternative sizes. Even when used in, for example the LIMA, the exact measurements stated in the text may vary depending on the application, the type of vessel (e.g., artery, vein, xenograft, synthetic graft), size of connector, shape of hole (e.g., incision, round) and/or sizes of vessels involved (e.g., 1 mm, 2 mm, 3 mm, 5 mm, aorta sized).

In some embodiments, one or more of the devices, generally sterilize, described above, are packaged and/or sold with an instruction leaflet, describing the device dimensions and/or situations for which the device should be applied. Also within the scope of the invention are surgical kits comprising sets of medical devices suitable for making anastomotic connections.

It should be appreciated that the above may be varied and still fall within the scope of the invention, for example, by changing the order of steps or by providing embodiments which include features from several described embodiments or by omitting features described herein. Section headings where are provided are intended for aiding navigation and should not be construed to limiting the description to the headings.

When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A leg compacting system for compacting inwards a group of legs of an anastomotic connector towards a central location thereof, comprising:
a coupler for coupling to a delivery system on which said connector is mounted; and
a plurality of leg confiners, said leg confiners configured to selectively move in an inwards direction and said confiners configured to have a resting compacted configuration in which a space of between 7 and 1 mm in width is maintained between the innermost leg contacting sections of said confiners such that a graft vessel suitable for mounting on said connector can be passed between said confiners.

2. A system according to claim 1, wherein said resting configuration is an innermost configuration.

3. A system according to claim 1, wherein said space is less than 5 mm in width.

4. A system according to claim 1, wherein said space is oval or circular.

5. A leg compacting system according to claim 1, configured to be selectively dismounted from said delivery system.

6. A leg compacting system according to claim 1, comprising a control for splitting at least a portion of said system for removal from said delivery system.

7. A leg compacting system according to claim 1, wherein said confiners move in a radial direction.

8. A leg compacting system according to claim 1, comprising a rotatable control which selectively moves said confiners in a radial direction.

9. A leg compacting system according to claim 1, wherein said system is permanently mounted on said delivery system.

10. A leg comprising system according to claim 1, wherein each confiner is configured to receive a single leg.

11. A leg comprising system according to claim 1, wherein said plurality of leg confiners are configured to release legs when moved outwards, said release being not simultaneous for all legs.

12. A leg compacting system for impacting inwards a group of legs of an anastomotic connector towards a central location thereof, comprising:
a coupler for coupling to a delivery system on which said connector is mounted; and
a plurality of leg confiners, configured to selectively move in an inward direction and to automatically engage said legs while moving inwards.

13. A leg compacting system according to claim 12, wherein each leg confiner is configured to receive a plurality of legs.

14. A leg compacting system according to claim 12, wherein each leg confiner is configured to receive a single leg.

15. A leg compacting sys according to claim 12, wherein said motion is radial.

16. A leg compacting system for compacting inwards a group of legs of an anastomotic connector towards a central location thereof, comprising:
at least one wire arranged to selectively move inwards, from a position outwards of the legs, thereby compacting the legs; and
a controller which is operative to selectively moving said wire.

17. A leg compacting system according to claim 16, wherein said wire is adapted to engage said legs near a hook section of the legs.

18. A leg compacting system according to claim 16, comprising at least two wires configured to compact the legs from two directions.

19. A leg compacting system according to claim 16, wherein said wire comprises a side wall adapted to prevent legs from slipping away from compacting.

20. A leg arranging device for use with a noose, comprising:
a body adapted to receive a delivery system on which a plurality of connector legs are mounted;
a noose receptacle arranged around an expected position of said legs, said receptacle including an inner block which selectively blocks said noose from leaving said receptacle to engage said legs; and
a control is operative removes said block.

21. A device according to claim 20, wherein said control is automatically activated to release said noose once a delivery system is inserted in said body and said legs are at said expected position.

22. A device according to claim 20, wherein said control is manually activated.

23. A device according to claim 20, comprising a noose controller which pre-stresses said noose to reduce its diameter.

24. A leg compacting system for compacting inwards a group of legs of an anastomotic connector towards a central location thereof, comprising:
at least one element adapted to compact a plurality of connector legs to a compacted configuration; and
at least controller configured to activate said element thereby releasing said legs,
wherein, said at least one element is configured to release certain connector legs before other ones of said legs as said element is activated.

25. A system according to claim 24, wherein said element is configured to first release legs that are on a long axis of an incision into which said legs are inserted for connection.

26. A system according to claim 24, wherein said at least one element defines a plurality of notches each configured for receiving at least one leg and wherein an end notch of said at least one element is configured to release a leg earlier than a leg held by a more central notch.

27. A leg compacting system for compacting inwards a group of legs of an anastomotic connector towards a central location thereof, comprising:
a cap adapted to mount on a delivery system, said cap comprising:
at least two sections adapted to form said cap;
each of said sections having a front plate defining a plurality of receptacles for legs; and
a control adapted to split said cap into said sections.

28. A method of using a noose-type compacting system, comprising:
mounting a noose on a plurality of connector legs;
compacting the legs using the noose;
inserting said compacted legs into an aperture in a blood vessel;
releasing and tightening said noose until a desired leg configuration is achieved; and
removing said noose.

29. A method according to claim 28, wherein removing said noose comprises cutting an extension of said noose.

30. A method according to claim 28, wherein mounting comprises:
arranging said noose around said legs, such that said noose is pre-stressed; and
releasing said noose to engage said legs.

31. An anastomosis connector delivery system, comprising:
a body having an end adapted to mount a connector thereon;
a handle adapted to be attached to said body at a plurality of different orientation positions relative to said body;
at least one control for deploying said connector, said handle being devoid of controls for deploying the connector.

32. A system according to claim 31, wherein said handle is a pistol grip.

33. A system according to claim 31, wherein said control is shaped as and moves as a trigger.

34. An anastomosis delivery system, comprising:
a body having a handle section and having a plurality of connector legs attached to one end thereof;
an activation control; and
a leg retraction and tearing mechanism,
wherein said control both retracts said legs relative to said body and tears said legs and wherein said control applies said retracting and said tearing without requiring movement of said handle by a user.

35. A delivery system according to claim 34, wherein said control releases a spring which provides said tearing.

36. A delivery system according to claim 34, wherein said body comprises a moving section and a stationary section, such that said retraction of said legs moves said moving section towards said legs and does not move said legs relative to said stationary section.

37. A delivery system according to claim 35, comprising a recoil absorber which absorbs at least part of a recoil of said spring prior to such recoil affecting said handle/

38. A sterile anastomosis connector leg manipulator, comprising:
a handle; and
a two pronged extension defining a receptacle between the prongs, said receptacle being sized to receive one leg of an anastomosis connector suitable for attaching a vessel of a diameter smaller than 4 mm, said prongs being thin enough to fit between adjacent legs of such a connector.

39. A leg arranging system for arranging legs of a blood vessel anastomotic connector, comprising:
a coupler for coupling to a delivery system on which said connector is mounted;
a body; and
a plurality of notches defined by said body, each of said notch configured to hold a single leg.

40. A leg arranging system according to claim 39, wherein said notches prevent said legs from crossing.

41. A leg arranging system according to claim 39, wherein said notches position said legs in an inwards compacted configuration.

42. A method of mounting a graft, comprising:
arranging a plurality of connector legs in a leg arranger, such that their relative positions are fixed in a plane perpendicular to a general orientation of said legs;
providing a graft between said legs; and
mounting said graft on said arranged legs.

43. A method according to claim 42, wherein said legs are arranged to have an inward compacted configuration.

44. A method of inserting a graft into a blood vessel, comprising:
arranging a plurality of connector legs in a leg arranger to a have a desired mutual positional relationship and an inward compacted configuration; and
inserting said arranged legs into an aperture of a blood vessel.

45. An anastomotic connector for blood vessels, comprising:
a ring; and
a plurality of legs arranged around said ring, wherein at least two legs at opposing sides of said ring are configured to bend radially out more than other of said legs.

46. An anastomotic connector for blood vessels, comprising:
a ring; and
a plurality of legs arranged around said ring, wherein at least two legs at opposing sides of said ring are configured to be stiffer than other of said legs.

47. An anastomotic connector kit, comprising:
a plurality of leg segments arranged in a generally circular configuration; and
a plurality f leg locking segments, each adapted to be locked to one leg,
wherein, wherein at least two legs at opposing sides of said circle are configured to be stiffer than other of said legs.

48. An anastomotic connector kit, comprising:
a plurality of leg segments arranged in a generally circular configuration; and
a plurality f leg locking segments, adapted to be locked to one leg,
wherein, wherein at least two legs at opposing sides of said circle are configured to bend radially out more than other of said legs.

49. A connector kit, comprising
a sterile package;
a connector having a plurality of forward legs; and
a band radially comprising said legs towards a center, within the sterile package.

50. A method of mounting a graft on a connector delivery system capsule, comprising:
axially splitting said capsule;
laying said graft in said capsule;
closing said capsule; and
mounting said capsule on a connector of said capsule.

51. Apparatus for mounting a graft on a spoilable graft capsule, comprising:
a splitable connector capsule;
a body including a receptacle large enough to hold a split capsule and including a slot in its side; and
a control which selectable opens said body so said capsule can open.

52. Apparatus according to claim 51, wherein said control actively splits said capsule.

53. Apparatus according to claim 51, wherein said body is adapted to radially compact legs of a connector of said capsule.

54. A method of graft attachment to a target vessel, comprising:
inserting a plurality of legs into an aperture in said target vessel;
releasing at least two of said legs so that said at least two legs stretch said aperture; and
completing said anastomosis.

55. A method according to claim 54, wherein said releasing comprises releasing to ends of an incision.

56. A method of graft attachment to a target vessel, comprising:
inserting a plurality of legs into an aperture in said target vessel;
mechanically retracting said legs relative to a body of a delivery system; and
mechanically tearing said legs,
wherein said retracting and said tearing occur without motion of said legs relative to said vessel.

57. A method of performing an anastomosis, comprising:
forming an opening a target vessel;
inserting a shunt into said target vessel to bypass said opening;
inserting a plurality of anastomosis connector legs into said aperture;
removing said shunt between said legs, while said legs are in said aperture; and
completing said anastomosis.
